SEQUENCE LISTING (8 sequences)

SEQ ID NO: 1 - DNA, 1881 bp, Homo sapiens
gcggaggcggctccggcgcccgctctgcccgccgctgggtccgaccgcgctcgccttccttgcagccgcgcctcggccccatggacgccctgtgcggttccggggagctcggctccaagttctgggactccaacctgtctgtgcacacagaaaacccggacctcactccctgcttccagaactccctgctggcctgggtgccctgcatctacctgtgggtcgccctgccctgctacttgctctacctgcggcaccattgtcgtggctacatcatcctctcccacctgtccaagctcaagatggtcctgggtgtcctgctgtggtgcgtctcctgggcggaccttttttactccttccatggcctggtccatggccgggcccctgcccctgttttctttgtcacccccttggtggtgggggtcaccatgctgctggccaccctgctgatacagtatgagcggctgcagggcgtacagtcttcgggggtcctcattatcttctggttcctgtgtgtggtctgcgccatcgtcccattccgctccaagatccttttagccaaggcagagggtgagatctcagaccccttccgcttcaccaccttctacatccactttgccctggtactctctgccctcatcttggcctgcttcagggagaaacctccatttttctccgcaaagaatgtcgaccctaacccctaccctgagaccagcgctggctttctctcccgcctgtttttctggtggttcacaaagatggccatctatggctaccggcatcccctggaggagaaggacctctggtccctaaaggaagaggacagatcccagatggtggtgcagcagctgctggaggcatggaggaagcaggaaaagcagacggcacgacacaaggcttcagcagcacctgggaaaaatgcctccggcgaggacgaggtgctgctgggtgcccggcccaggccccggaagccctccttcctgaaggccctgctggccaccttcggctccagcttcctcatcagtgcctgcttcaagcttatccaggacctgctctccttcatcaatccacagctgctcagcatcctgatcaggtttatctccaaccccatggccccctcctggtggggcttcctggtggctgggctgatgttcctgtgctccatgatgcagtcgctgatcttacaacactattaccactacatctttgtgactggggtgaagtttcgtactgggatcatgggtgtcatctacaggaaggctctggttatcaccaactcagtcaaacgtgcgtccactgtgggggaaattgtcaacctcatgtcagtggatgcccagcgcttcatggaccttgcccccttcctcaatctgctgtggtcagcacccctgcagatcatcctggcgatctacttcctctggcagaacctaggtccctctgtcctggctggagtcgctttcatggtcttgctgattccactcaacggagctgtggccgtgaagatgcgcgccttccaggtaaagcaaatgaaattgaaggactcgcgcatcaagctgatgagtgagatcctgaacggcatcaaggtgctgaagctgtacgcctgggagcccagcttcctgaagcaggtggagggcatcaggcagggtgagctccagctgctgcgcacggcggcctacctccacaccacaaccaccttcacctggatgtgcagccccttcctggtgaggcttggcacagggctgggtccctgcctccagggctctgggtgcccaggcatggccagggctcattggactctaccctgacaccacctccacgctgctcaggtgaccctgatcaccctctgggtgtacgtgtacgtggacccaaacaatgtgctggacgccg

SEQ ID NO: 2 - DNA, 898 bp, Homo sapiens
tgcccccgatgaggaccaagggcacctggaggacagctggaccgcgttggaaggtgcagaggataaggaggcactgctgattgaagacacactcagcaaccacacggatctgacagacaatgatccagtcacctatgtggtccagaagcagtttatgagacagctgagtgccctgtcctcagatggggagggacagggtcggcctgtaccccggaggcacctgggtccatcagagaaggtgcaggtgacagaggcgaaggcagatggggcactgacccaggaggagaaagcagccattggcactgtggagctcagtgtgttctgggattatgccaaggccgtggggctctgtaccacgctggccatctgtctcctgtatgtgggtcaaagtgcggctgccattggagccaatgtgtggctcagtgcctggacaaatgatgccatggcagacagtagacagaacaacacttccctgaggctgggcgtctatgctgctttaggaattctgcaagggttcttggtgatgctggcagccatggccatggcagcgggtggcatccaggctgcccgtgtgttgcaccaggcactgctgcacaacaagatacgctcgccacagtccttctttgacaccacaccatcaggccgcatcctgaactgcttctccaaggacatctatgtcgttgatgagcgcttctatgcagccacatcacggcaactgaagcggctggaatcagtcagccgctcacctatctactcccacttttcggagacagtgactggtgccagtgtcatccgggcctacaaccgcagccgggattttgagatcatcagtgatactaaggtggatgccaaccagagaagctgctacccctacatcatctccaaccggtggctgagc

SEQ ID NO: 3 - DNA, 20 bp, Homo sapiens
taggtccctctgtcctggct

SEQ ID NO: 4 - DNA, 20 bp, Homo sapiens
gggacccagccctgtgccaa

SEQ ID NO: 5 - DNA, 25 bp, Homo sapiens
acacttccctgaggctgggcgtcta

SEQ ID NO: 6 - DNA, 25 bp, Homo sapiens
ctgcatagaagcgctcatcaacgac

SEQ ID NO: 7 - DNA, 20 bp, Homo sapiens
acggatttggtcgtattggg

SEQ ID NO: 8 - DNA, 20 bp, Homo sapiens
cgctcctggaagatggtgat

(19) United States
(12) Patent Application Publication
(10) Pub. No.: US 2019/0175639 A1
(43) Pub. Date: Jun. 13, 2019

(54) APPLICATION OF SPLICING VARIANT ABCC3-013 MRNA FOR MANUFACTURING CLOPIDOGREL RESISTANCE TEST KIT AND THE TEST KIT

(71) Applicant: NANJING FIRST HOSPITAL, NANJING (CN)

(72) Inventors: Hong-Guang XIE, NANJING (CN); Weiqiang LIU, NANJING (CN); Yuqin PAN, NANJING (CN); Ping YANG, NANJING (CN); Qian YIN, NANJING (CN); Qiongyu MI, NANJING (CN)

(73) Assignee: NANJING FIRST HOSPITAL

(21) Appl. No.: 16/231,603

(22) Filed: Dec. 24, 2018

Related U.S. Application Data
(63) Continuation of application No. PCT/CN2017/090451, filed on Jun. 28, 2017.

(30) Foreign Application Priority Data
Jun. 29, 2016 (CN) 201610489596.5

(51) Int. Cl. A61K 31/7105; C12Q 1/6827; C12Q 1/6816; C12Q 1/6832; C12Q 1/6876

(52) U.S. Cl. CPC A61K 31/7105; C12Q 1/6827; C12Q 1/6876; C12Q 1/6832; C12Q 1/6816

(57) ABSTRACT

An application of an ABCC3-013 mRNA splicing variant for manufacturing a testing kit for clopidogrel resistance is provided. Part of the nucleotide sequences of the ABCC3-013 mRNA is described as SEQ ID No. 2. The test kit can measure an in vivo (such as peripheral leukocytes) expression levels of the ABCC3-013 mRNA of a patient to determine clopidogrel resistance, thereby providing a new method for diagnostics of clopidogrel resistance, which would help guide personalized therapy of clopidogrel in patient care. An all new test kit is provided for assay of clopidogrel resistance, characterized by an AUC of 0.674 (P=0.0018), a sensitivity of 52%, and a specificity of 86% for its performance in clinical settings, all of which are significantly improved compared with CYP2C19 genotyping that is recommended by US FDA and CPIC for the same purpose.

### CROSS-REFERENCE TO RELATED APPLICATIONS

This application is a continuation of International Patent Application No. PCT/CN2017/090451 with a filing date of Jun. 28, 2017, designating the United States, now pending, and further claims priority to China Patent Application No. 201610489596.5 with a filing date of Jun. 29, 2016. The content of the aforementioned applications, including any intervening amendments thereto, are incorporated herein by reference.

### TECHNICAL FIELD

The present disclosure relates to the technical field of a novel testing kit for detecting drug resistance, and particularly of manufacturing a potentially commercial kit for assay of clopidogrel resistance in humans.

### BACKGROUND OF THE PRESENT INVENTION

Clopidogrel, as a marketed platelet inhibitor, is one of the most frequently prescribed medicines to prevent recurrent thrombotic or ischemic events or in-stent thrombosis for patients with coronary artery disease and undergoing percutaneous coronary intervention (PCI), followed by drug-eluting stents. The dual antiplatelet therapy (generally combination of clopidogrel and aspirin) is considered as gold standard therapy in patients with acute coronary syndrome (ACS) and/or receiving PCI with coronary stenting. Clopidogrel resistance is defined as inadequate or poor platelet inhibition in patients taking clopidogrel, consequently leading to therapy failure. In clinical settings, the prevalence of clopidogrel resistance is estimated to be 10%-40% in patients treated with clopidogrel, which varies by the assay of clopidogrel resistance.

The existing assay of platelet aggregation and thromboelastography test could help evaluate clopidogrel resistance to a certain extent. However, platelet aggregation test exhibits poor replication and lack of widely recognized assay and evaluation criteria for platelet aggregation. Therefore, novel assay of detection of platelet reactivity has being explored.

Currently, there are inconsistent recommendations from the AHA (American Heart Association)/ACC (American College of Cardiology) versus US Food and Drug Administration (FDA) and CPIC (Clinical Pharmacogenetics Implementation Consortium) for routine use of CYP2C19 genotyping as a predictor of clopidogrel resistance in clopidogrel-treated patients. CYP2C19 genotyping detection based on peripheral blood leukocyte DNA reflects approximately 10% of the overall variations in clopidogrel antiplatelet efficacy in patient care, and thus its value in diagnostics or prediction is very limited as indicated by an area under the ROC (receiver operator characteristic) curve of 0.617 (P<0.001), accompanied by a sensitivity of 30.4% for identifying the individuals at risk of developing clopidogrel resistance and a specificity of 93%. Moreover, the mechanisms behind clopidogrel resistance are still unclear, and therefore there is still the urgent need to identify novel biomarkers to predict or diagnose clopidogrel resistance for patient care.

### SUMMARY OF PRESENT INVENTION

The objective of this disclosure is to provide an application of a novel clopidogrel resistance biomarker in manufacturing a test kit for clopidogrel resistance, which can help guide personalized clinical therapy of clopidogrel and improve its therapeutic efficacy.

Another objective of this disclosure is to provide a novel test kit for clopidogrel resistance in clinical settings.

The present disclosure provides an application of ABCC3-013 mRNA (mRNA splicing variant itself) in manufacturing clopidogrel resistance test kit, and nucleotide sequence of the ABCC3-013 mRNA is provided in SEQ ID No. 2.

The disclosure further provides the clopidogrel resistance test kit, in which the major components include, but not limited to, specific primers and probes corresponding to the ABCC3-013 mRNA with the nucleotide sequence described as SEQ ID No. 2.

According to another schematic embodiment, the specific primers in the test kit comprise primers described as SEQ ID No. 5 and SEQ ID No. 6.

According to another schematic embodiment, the test kit further comprises a TRIzol reagent, chloroform, isopropanol, DEPC-treated water, an Oligo (dT) primer, a Random 6-mers primer, reverse transcriptase (RT) mix, RNase-free dH₂O, RT buffer, Taq enzyme, dNTPs, Mg²⁺, SYBR Green I, and ROX reference dye.

Multidrug resistance-associated protein (MRP) 3 is the product of the gene expression of ABCC3 (ATP binding cassette, subfamily C, member 3). Clinical research studies have demonstrated that a higher expression level of MRP3 is associated with resistance of cancer cells to some cancer chemotherapy drugs. Because the splicing variants could happen at the transcriptional level of ABCC3 mRNA, ABCC3-013 is such abnormal mRNA splicing variant caused by point mutation of the ABCC3 gene. In this study, ABCC3-013 variant is observed to enhance the efflux transport activity mediated by MRP3 protein. The clinical research studies have further confirmed that patients resistant to clopidogrel exhibit significantly higher expression levels of ABCC3-013 mRNA than those responsive to clopidogrel.

The present disclosure provides a novel test kit for assay of clopidogrel resistance for patient care through the measurement of the expression levels of the ABCC3-013 mRNA extracted from peripheral blood leukocytes. The results demonstrated that the test kit has an AUC (area under the ROC curve) of 0.674 (P=0.0018), with a sensitivity of 52% and a specificity of 86%, all of which are significantly improved compared with CYP2C19 genotyping (see above [0005] for details).

### DESCRIPTION OF THE FIGURES

The following accompanied figures are only for the purpose of schematic illustration and explanation of the present disclosure, but not limit to the scope of the disclosure itself.

FIG. 1 is a ROC (receiver operator characteristic) curve describing how to use ABC3-013 mRNA slicing variant to diagnose or predict clopidogrel resistance;

FIG. 2 is profiling of protein expression as marked by EGFP in different groups;

FIG. 3 is expression profiling of ABCC3-002 and ABCC3-013 in three stably transfected cell lines;

FIG. 4 is used to directly reflect efflux transport capability (as measured with fluorescence intensity) of three different stably transfected cell lines; and

FIG. 5 is used to direct comparison of the difference in ABCC3-013 mRNA relative expression levels in responders (Q1) versus non-responders (Q4) to clopidogrel in real-world clinical settings.

### DETAILED DESCRIPTION OF PREFERRED EMBODIMENTS

In order to make the technical characteristics, objectives and effects in the present disclosure clearer, the following embodiments will be further illustrated for details.

### Embodiment 1: Application of ABCC3-013 mRNA in Manufacturing Clopidogrel Resistance Test Kit

A test kit for clopidogrel resistance, disclosed in the present disclosure, comprises TRIzol reagent, chloroform, isopropanol, DEPC-treated water, an Oligo (dT) primer, a random 6-mers primer, PrimeScript™ RT enzyme mix, RNase-free dH₂O, PrimeScript™ buffer, Taq (enzyme), dNTPs, Mg²⁺, SYBR Green I (SYBR Premix Ex Taq™), and ROX reference dye, and specific primers are described as SEQ ID No. 5 and SEQ ID No. 6, both of which can specifically hybridize ABCC3-013 mRNA with part of nucleotide sequence as shown in SEQ ID No. 1.

In the methodology, the test kit comprises the two major steps: extraction of total RNA of peripheral blood leukocytes from patients, and detection of ABCC3-013 mRNA relative expression levels by qRT-PCR. For details, the technical performances are described as follows:

### 1. Extraction of Total RNA of Peripheral Leukocytes

1) Adding 750 μl of TRIzol reagent to 250 μl of whole blood;

2) Shaking 3-5 times, thoroughly mixing with a pipette, and transferring the mixed liquid to a 1.5-ml EP tube;

3) Leaving to stand for 10 min at room temperature;

4) Adding 200 μl of chloroform, mixing vigorously or performing vortex oscillation for 15 s, and leaving to stand for 5 min at room temperature;

5) Centrifugation at a speed of 12,000×g, at temperature of 4° C. for 15 min;

6) Transferring the supernatant containing total RNA into a fresh 1.5-ml EP tube, adding 500 μl of isopropanol, inverting and mixing, and then letting stand for 10 min at room temperature;

7) Centrifugation at a speed of 12,000×g, at temperature of 4° C. for 10 min, and removing the supernatant;

8) Adding 1 ml of 75% ethanol prepared with DEPC-treated water, and performing vortex oscillation;

9) Centrifugation at a speed of 7,500×g, at temperature of 4° C. for 5 min, and remove the supernatant;

10) Centrifugation at a speed of 7,500×g, at temperature of 4° C. for 5 min, and removing excess liquid; and

11) Re-dissolving with 20 μl of DEPC-treated water after the RNA is slightly dried; and storing at −80° C. until analysis.

### 2. RNA Quantification and Purity Determination

Adding 1 μl of the above RNA to 99 μl of dH₂O to dilute RNA for 100 times, measuring optical density (OD) values at 260 nm and 280 nm respectively (i.e., OD260 and OD280, respectively) and calculating the ratio of OD260 to OD280 to determine the purity of the RNA samples obtained. The ratio is:

<1.6, indicating that the RNA samples contain proteins remained;

>1.9, indicating that the purity of RNA sample is high; or

1.6-1.9, indicating that the RNA sample contains remained DNA to a certain extent. The RNA concentrations are calculated according to the OD260 value and the formula shown below:

C(μg/μl)=30×OD260×D/1000

where C refers to the RNA concentrations of samples, OD260 is the OD values of RNA measured at 260 nm, and D is the multiple of dilution. Finally, adjusting the RNA concentrations of samples to 1 μg/μl with DEPC-treated water based on their calculated actual concentrations.

### 3. Reverse Transcription from RNA to cDNA

Preparing a 20-μl reaction system by adding, each of the components indicated in Table 1:

TABLE 1
Reaction system of reverse transcription

| Reagent | volume |
|---|---|
| RNA (1 μ/μl) | 1 μl |
| Oligo dT primer (50 μM) | 1 μl |
| Random 6-mers (100 μM) | 1 μl |
| PrimeScript ™ RT Enzyme Mix | 1 μl |
| 5 × PrimeScript ™ buffer | 4 μl |
| RNase-free dH₂O | 12 μl |

The amplification reaction is carried out at 37° C. for .15 min and at 85° C. for 5 s. The obtained cDNA is stored at 4° C.

### 4. Real-time Quantitative PCR (also known as qRT-PCR)

Using qRT-PCR (ABI 7500, USA) and a SYBR Premix Ex Taq™ qRT-PCR reagent (TaKaRa Biotech Co., Ltd, Dalian, China) to detect the ABCC3-013 cDNA levels. The sequences of primers are showed in Table 2, and a 20-μl reaction system is showed in Table 3.

TABLE 2
Primer sequences

| Name | Sequence |
|---|---|
| ABCC3-013 | Forward primer: SEQ ID No. 5; |
| | Reverse primer: SEQ ID No. 6 |
| GAPDH | Forward primer: SEQ ID No. 7; |
| | Reverse primer: SEQ ID No. 8 |

TABLE 3
Reaction system of qRT-PCR

| Reagent | volume |
|---|---|
| cDNA (sample) | 1 μl |
| Forward primer (10 μM) | 0.8 μl |
| Reverse primer (10 μM) | 0.8 μl |
| SYBR Premix Ex Taq ™ | 10 μl |
| ROX Reference Dye | 0.4 μl |
| dH₂O | 7 μl |

The amplification reaction is carried out as follows: at 95° C. for 30s, and subsequently at 95° C. for 5 s, 60° C. for 30 s, and 72° C. for 30 s, for 40 cycles in total. The results of qRT-PCR are expressed with the number of PCR cycles (Ct value) that have crossed an arbitrarily chosen signal threshold in the log phase of the amplification curve. The relative expression level of ABCC3-013 is calculated by using the 2^{-ΔΔCt} method, where ΔCt=Ct_{(ABCC3-013)}−Ct_{(GAPDH)}.

A total of 100 patients with coronary artery disease (diagnosed by coronary angiography) who underwent PCI and drug-eluting stenting were prescribed for dual antiplatelet therapy strategy. Their maintenance doses were 75 mg clopidogrel and 100 mg aspirin per day for at least 6 months. After that, two tubes of 3-ml blood were taken from fasting patients before checking again of their antiplatelet therapeutic efficacy with coronary angiography. One tube is blue cap tube in which 3.2% sodium citrate anticoagulant was added to determine ADP-induced whole-blood platelet aggregation, and the other is red cap tube in which EDTA anticoagulant was used to extract total RNA of peripheral blood leukocytes.

The patients enrolled were divided into two groups according to the extent of inhibition of whole-blood platelet aggregation by clopidogrel. One was resistance-negative group, including 50 individuals who were sensitive or normal responders to clopidogrel, characterized by their platelet aggregation of 0 -1 ohm, and the other was a resistance-positive group, including 50 patients hypo- or non-sensitive to clopidogrel platelet responses, with their platelet aggregation of more than 1.0 ohm measured.

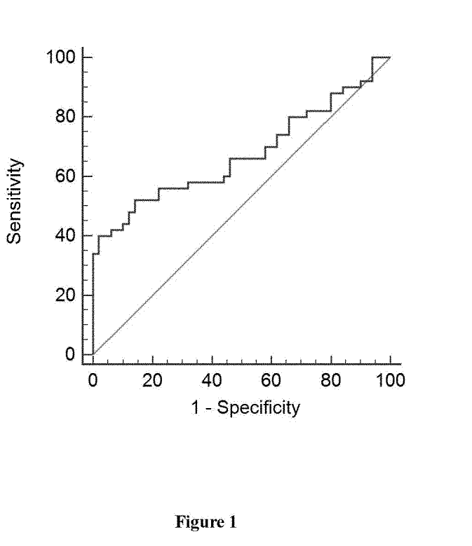

Peripheral leukocyte RNA of the patients was extracted according to the above method, and their ABCC3-013 mRNA expression levels were detected by qRT-PCR. In the present disclosure, 50 responders to clopidogrel was considered as a negative group, while 50 less- or non-responders to clopidogrel was considered as a positive group. Their expression levels of ABCC3-013 mRNA were detected by qRT-PCR approach, and an ROC curve of using ABCC3-013 mRNA expression to diagnose clopidogrel resistance was established (as shown in FIG. 1). The results demonstrated that the AUC (area under the ROC curve) is 0.674 (P=0.0018), with a sensitivity (also known as true-positive percentage) of 52% and a specificity (true-negative percentage) of 86%, accompanied by an optimal cutoff value of diagnosis of clopidogrel resistance is more than 7.029.

### Embodiment 2: Experimental Studies

The ABCC3-013 mRNA is an abnormal mRNA splicing variant caused by a point mutation of ABCC3 gene. In the following experiments, another splicing variant ABCC3-002 is used as a control to head-to-head compare the differences in their efflux transport capability in vitro.

1. Cell culture: HepG2 cells were cultured in high-glucose DMEM medium containing 10% inactivated fetal bovine serum, 50 U/ml penicillin, and 50 μg/ml streptomycin in an incubator set at a temperature of 37° C. and at a saturated humidity CO₂ content of 5%.

2. Construction of the plasmids.

The ABCC3-002 (SEQ ID No. 1) and ABCC3-013 (SEQ ID No. 2) fragments were first chemically synthesized, then ABCC3-002-EGFP-and ABCC3-013-EGFP were synthesized, and random fragments were synthesized as a control. Each of the fragments synthesized was cloned into a TV5 vector. The recombinant vectors were named as TV5, ABCC3-002, ABCC3-013, TV5-EGFP, ABCC3-002-EGFP, and ABCC3-013-EGFP, respectively. The recombinant plasmids were verified by direct sequencing. The sequencing results indicated that the recombinant plasmids TV5, ABCC3-002, ABCC3-013, TV5-EGFP, ABCC3-002-EGFP, and ABCC3-013-EGFP were constructed successfully.

3. Observation of the expression of proteins containing EGFP after HepG2 cells were transfected with each of recombinant plasmids.

HepG2 cells were seeded in 6-well plates at a density of 3×10⁵ and incubated overnight. Recombinant plasmids TV5-EGFP, ABCC3-002-EGFP, and ABCC3-013-EGFP were transfected into HepG2 cells by liposome Lipofectamine 2000 (Invitrogen, Carlsbad, Calif., USA). After transfection for 48 h, the expression of proteins containing EGFP by fluorescence inverted microscope.

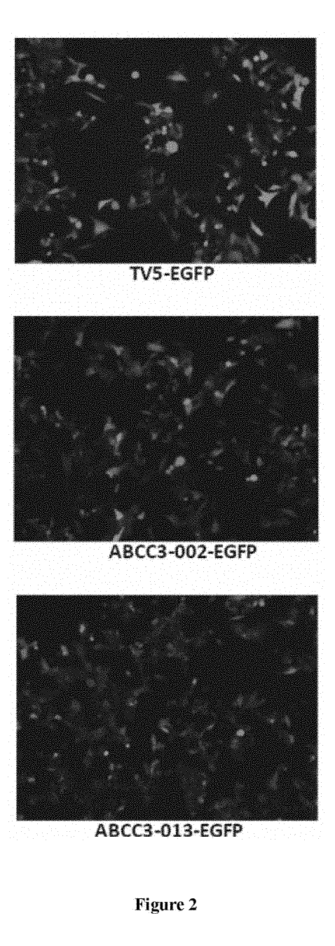

As shown in FIG. 2, EGFP protein was expressed in all groups of cells, indicating the successful construction of recombinant plasmids expressing ABCC3-002 and ABCC3-013, respectively.

4. Screening for stable transfection of HepG2 cell line.

HepG2 cells were seeded in 6-well plates at a density of 3×10⁵ and incubated overnight; Recombinant plasmids TV5, ABCC3-002 and ABCC3-013 were transfected into HepG2 cells by liposome Lipofectamine 2000 (Invitrogen, Carlsbad, Calif., USA). After transfection for 48 h, transfected HepG2 cells were diluted and inoculated in 96-well plates. 500 μg/ml G418 (Gibco BRL, Grand Island, N.Y., USA) were used to screen for monoclonal cell lines. A positive monoclone was selected for amplification, and a stably transfected monoclonal cell line was identified by RT-PCR. HepG2 cells stably transfected with recombinant plasmids TV5, ABCC3-002, and ABCC3-013 were named as TV5 cells, 002 cells, and 013 cells, respectively.

5. Detection of the expression of ABCC3-002 and ABCC3-013 in stable transfection by using qRT-PCR.

Extracting total RNA of different stable transfection cell lines using TRIzol reagent, and using reverse transcription to make RNA into cDNA with the system showed in Table 4.

TABLE 4
Reaction system of reverse transcription

| Reagent | volume |
|---|---|
| RNA (1 μg/μl) | 1 μl |
| Oligo dT primer (50 μM) | 1 μl |
| Random 6-mers (100 μM) | 1 μl |
| PrimeScript ™ RT Enzyme Mix | 1 μl |
| 5 × PrimeScript ™ buffer | 4 μl |
| RNase-free dH₂O | 12 μl |

Using qRT-PCR (ABI 7500, USA) and a SYBR Premix Ex Taq™ qRT-PCR reagent (TaKaRa Biotech Co., Ltd, Dalian, China) to detect the expression levels of ABCC3-002 and ABCC3-013. The sequences of primers used are showed in Table 5, and a 20-μl reaction system is showed in Table 6.

TABLE 5
Primer sequences

| Name | Sequence |
|---|---|
| ABCC3-013 | Forward primer: SEQ ID No. 5; |
| | Reverse primer: SEQ ID No. 6 |
| ABCC3-002 | Forward primer: SEQ ID No. 3; |
| | Reverse primer: SEQ ID No. 4 |

TABLE 6
Reaction system of qRT-PCR

| Reagent | volume |
|---|---|
| cDNA sample | 1 μl |
| Forward primer (10 μM) | 0.8 μl |
| Reverse primer (10 μM) | 0.8 μl |
| SYBR Premix Ex Taq ™ | 10 μl |
| ROX Reference Dye | 0.4 μl |
| dH₂O | 7 μl |

The amplification reaction is carried out as follows: at 95° C. for 30 s and at 95° C. for 5 s, 60° C. for 30 s, and 72° C. for 30 s, for 40 cycles in total. The results of qRT-PCR are expressed with the number, of PCR cycles (Ct value) when the fluorescence signal in each reaction tube reaches a preset threshold. The relative expression levels of ABCC3-002 and ABCC3-013 are calculated by using the 2^{-ΔΔCt} method, where ΔCt=Ct_{(ABCC3-002 or ABCC3-013)}−Ct_{(GAPDH)}.

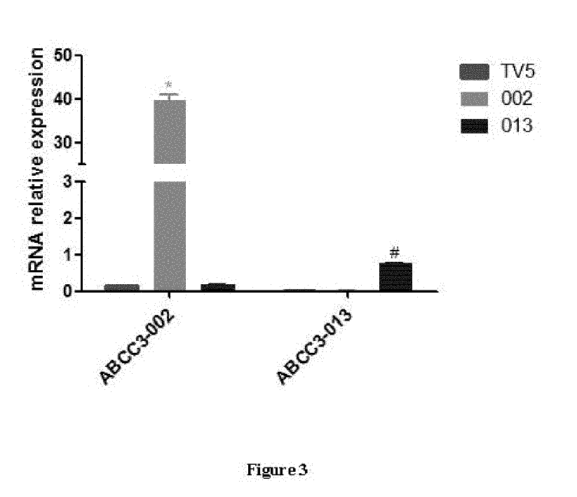

As shown in FIG. 3 that compares the expression levels of ABCC3-002 mRNA splice variant in 3 groups of cells the expression level in 002 cells was significantly higher than that in control TV5 cells (P<0.05), but there is no significant difference between the expression level of the 013 cells and that of control TV5 cells. Similarly, the expression level of ABCC3-013 splicing variant in 013 cells was significantly higher than that in control TV5 cells (P<0.05), but no significant difference existed between the expression level of 002 cells and that of control TV5 cells.

6. Detection of transport activity

5-Carboxyfluorescein diacetate (5-CFDA) is converted to the fluorescent anion 5-carboxyfluorescein (5-CF) by intracellular esterase hydrolysis, and therefore the latter is used as a fluorescent probe. In addition, MRP proteins expressed on the cell membrane, including MRP3, can transport 5-CF from inside cells to outside, and therefore, the intracellular accumulation of 5-CF can be used to reflect the presence or absence of the MRP pump activity in ABCC3-002 or ABCC3-013 cells.

The TV5 cells, 002 cells, and 013 cells were seeded in 6-well plates at a density of 1×10⁶ cells/well, and incubated overnight at 37° C. After incubating for 24 h, washing up for 3 times with PBS, and adding 5-CFDA (5 μM, BD Biosciences, San. Jose, Calif., USA) and incubating for 30 min. Washing up by PBS, and then the fluorescence intensity (measured as a.u.) of fluorescent anion 5-CF can be detected by flow cytometry, and the mean fluorescence intensity (MFI) of each group was obtained.

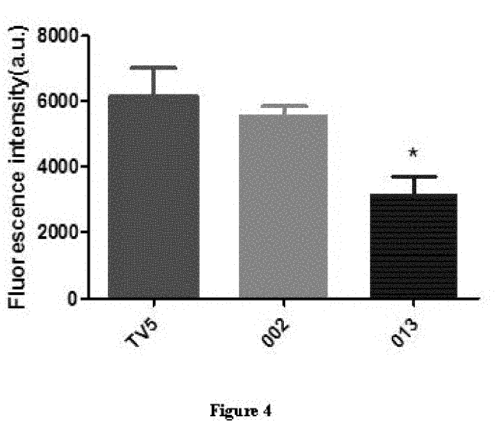

As shown in FIG. 4, the MFI of 5-CF in the 013 cells was significantly lower than that of the control TV5 cells (P<0.05), suggesting that the ABCC3-013 splice variant can enhance the MRP-mediated efflux transport function. In other words, ABCC3-013 mRNA splicing variant is a gain-of-function event.

Clopidogrel glucuronide conjugate is identified as a substrate of MRP3 in humans, and therefore efflux transport of this conjugate would be increased in the ABCC3-013 cells due to enhanced efflux function. Clopidogel glucuronide conjugation is the main metabolic pathway for clopidogrel in humans. Hepatic metabolism of clopidogrel will be accelerated because of enhanced efflux transport of clopidogrel glucuronide conjugate in the ABCC3-013 cells, resulting in a decrease in the concentrations of clopidogrel in hepatocytes. Therefore, the amount of clopidogrel diverted to formation of clopidogrel active metabolite would be decreased, leading to attenuated antiplatelet effect of clopidogrel and consequently clopidogrel resistance.

7. Relevant clinical research study.

A total of 100 patients undergoing PCI and receiving drug-eluting stents had taken 75 mg clopidogrel and 100 mg aspirin per day for more than 6 months. Before checking again with coronary angiography for their antiplatelet efficacy, two tubes of 3-ml blood (blue-cap tube or red-cap tube) were taken from each of fasting patients for determination of ADP-induced whole-blood platelet aggregation (3.2% sodium citrate as anticoagulant) and extraction of total RNA of the peripheral leukocytes (EDTA as anticoagulant).

Patients were divided into two groups according to the extent of inhibition of platelet aggregation by clopidogrel. Group 1 (Q1): including 50 patients sensitive to clopidogrel platelet responses (as measured with 0-1 ohm); and group 2 (Q4): including 50 patients resistant to clopidogyel platelet responses (as measured with more than 10 ohm).

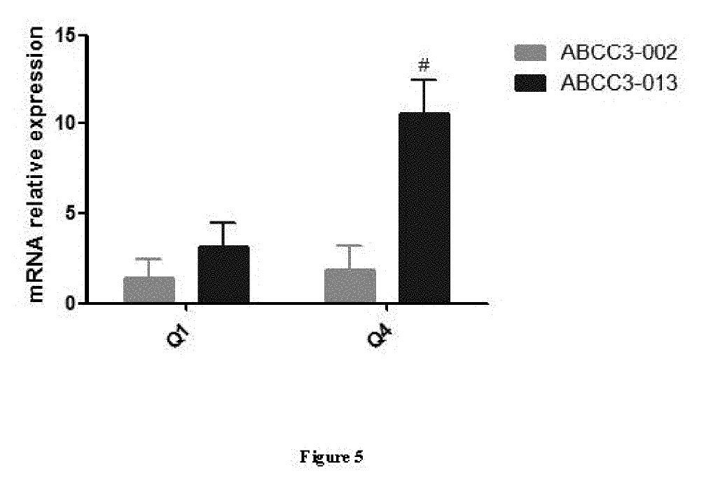

Then RNA of peripheral blood leukocytes of the patients divided in Q1 and Q4 groups was extracted, respectively, and the mRNA levels of ABCC3-002 and ABCC3-013 were detected by qRT-PCR, referring to the methods in Embodiment 1, where the sequence of primer ABCC3-002 is showed in Table 5. As shown in FIG. 5, mRNA expression levels of ABCC3-013 in Q4 group is significantly higher than those in Q1 group. The differences between the two groups were statistically significant (P<0.05).

Statistical analysis used in the above experiments is described as follows. Experimental data are expressed by mean value±SD. The difference between the means of each group is compared by unpaired Student's t test, and the repeated measurement data is performed by ANOVA (GraphPad Prism version 5, USA), and P <0.05 is considered statistically significant.

In the present disclosure, “schematic” means “serving as an example, instance, or illustration” and any embodiment described herein as “schematic” should not be construed as a more preferred or advantageous embodiment.

In the present disclosure, words like “equal” and “identical” are not limitations in strict mathematics and/or geometries. They should include errors that can be understood by those of ordinary skill in the art or tolerated in production and use. Unless otherwise stated, the range of value herein includes not only the entire scope between the two endpoints but also any sub-ranges contained therein.

It should be understood that, although the description has been described in terms of various embodiments, it is not intended that each embodiment only comprises one technical solution. The objective of this describing way is only to make disclosure clearer. The disclosure should be considered as a whole by those of ordinary skill in the art, and embodiments in different embodiments could be combined appropriately to form other embodiments that can be understood by those of ordinary skill in the art.

The series of detailed descriptions set forth above are merely for illustrating feasible embodiments in detail, and not for limiting the protecting scope of present invention. Any equivalent embodiments or changes without departing from the technical spirits of the invention, such as combinations; divisions or repetitions of features, are deemed to be included in the protection scope of the invention.

We claim:

1. An application of ABCC3-013 mRNA for manufacturing a clopidogrel resistance test kit, wherein the nucleotide sequence of the ABCC3-013 mRNA is described as SEQ ID No. 2.

2. A clopidogrel resistance test kit comprising specific primers, probes, and/or chips corresponding to the ABCC3-013 mRNA, wherein the nucleotide sequence of the ABCC3-013 mRNA is described as SEQ ID No. 2.

3. The test kit according to claim 2, wherein the specific primers comprise primers described in SEQ ID No. 5 and SEQ ID No. 6.

4. The test kit according to claim 2, further comprising TRIzol reagent, chloroform, isopropanol, DEPC-treated water, an Oligo dT primer, a random 6-mers primer, reverse transcriptase enzyme mix, RNase-free dH₂O, reverse transcriptase enzyme buffer, Taq (enzyme), dNTPs, Mg²⁺, SYBR Green I, and ROX reference dye.
